(19) United States
(12) Patent Application Publication
(10) Pub. No.: US 2011/0287097 A1
(43) Pub. Date: Nov. 24, 2011

(54) PHARMACEUTICAL COMPOSITIONS COMPRISING ROPINIROLE

(75) Inventors: Hemanth Prakash Joshi, Gadag (IN); Narayanan Badri Vishwanathan, Chennai (IN)

(73) Assignees: DR. REDDY'S LABORATORIES, INC., Bridgewater, NJ (US); DR. REDDY'S LABORATORIES LIMITED, Hyderabad 500 016, Andhra Pradesh (IN)

(21) Appl. No.: 12/673,051

(22) PCT Filed: Aug. 14, 2008
(86) PCT No.: PCT/US2008/073118

§ 371 Date: Feb. 11, 2010

Related U.S. Application Data
(60) Provisional application No. 60/985,479, filed on Nov. 5, 2007.
(60) Provisional application No. 61/026,170, filed on Feb. 5, 2008.

(30) Foreign Application Priority Data
Aug. 14, 2007 (IN) 1817/CHE/2007
Dec. 17, 2007 (IN) 3005/CHE/2007

(51) Int. Cl. A61K 31/404; A61K 9/58; A61P 25/16; A61K 9/22

(52) U.S. Cl. 424/462; 514/418; 424/468

(57) ABSTRACT

Controlled release pharmaceutical compositions comprising ropinirole or pharmaceutically acceptable salts thereof, wherein embodiments have ropinirole embedded in a matrix comprising one or more polymers, and the composition in monolithic form or in multiparticulate form.

The present invention relates to pharmaceutical compositions comprising ropinirole, including pharmaceutically acceptable salts thereof. More specifically, the present invention relates to controlled release pharmaceutical compositions comprising ropinirole, including pharmaceutically acceptable salts thereof, for therapeutic purposes, and methods of preparing the same.

Ropinirole is a dopamine D₂ agonist used in the treatment of Parkinson's disease. The precise mode of action for anti-Parkinson's is believed to be stimulation of post-synaptic dopamine D₂ type receptors in the caudate-putamen in the brain. Earlier methods for treating Parkinsonism typically involved the use of levodopa and patients undergoing treatment with levodopa have frequently experienced severe therapeutic response fluctuations, possibly resulting from decreased capacity to store newly synthesized dopamine within the brain as a consequence of the progressive degeneration of dopaminergic nerve terminals. Ropinirole has been developed as a drug which overcomes the limitations associated with levodopa therapy. Additionally, ropinirole has been identified as a more specific dopamine D₂ agonist than other dopamine agonists such as bromocriptine and pergolide.

Ropinirole has a chemical name 4-[2-(dipropylamino)ethyl]-1,3-dihydro-2H-indol-2-one (hereafter referred by its adopted name “ropinirole”), a molecular formula C₁₆H₂₄N₂O and is represented by structural Formula I.

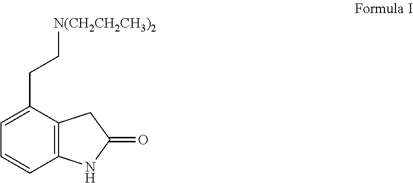

The hydrochloric acid salt, ropinirole monohydrochloride, is the active ingredient in a product that is approved for sale in many countries for the treatment of Parkinson's disease (PD) and restless leg syndrome (RLS) under the trade name REQUIP™ and manufactured by GlaxoSmithKline (GSK), as an immediate release tablet formulation for oral administration. GSK has also developed an oral controlled release tablet formulation of ropinirole marketed as REQUIP™ CR and its marketing application was recently approved in the U.S. This controlled release product was also approved for sale in several other countries including France, Canada, Slovakia, Slovenia, Latvia and Estonia for the treatment of PD. It has also been disclosed as being of potential use in the treatment of a variety of other conditions such as fibromyalgia, acute CNS injury, various sleep related disorders such as apneas, hypopneas and snoring events, and chronic fatigue syndrome.

Ropinirole is disclosed in U.S. Pat. Nos. 4,452,808 and 4,588,740, where it is said to have antihypertensive and anti-anginal properties. U.S. Pat. Nos. 4,824,860 and 4,912,126 disclose that ropinirole is a potent CNS active non-ergot dopamine receptor antagonist.

U.S. Patent Application Publication No. 2004/0157910 A1 describes a method of treatment or prophylaxis of restless leg syndrome using ropinirole or a pharmaceutically acceptable salt or solvate thereof.

Ropinirole is rapidly absorbed from the gastrointestinal tract and peak plasma concentration is reached within 1-2 hours after administration of an immediate release tablet. The absolute systemic bioavailability is 55%, indicating the presence of first-pass metabolism. The mean plasma elimination half-life is approximately 6 hours and hence the drug requires frequent dosing in order to maintain therapeutic levels throughout the day, which leads to fluctuations in plasma concentrations of ropinirole and possible undesirable side effects. Hence, there is a need for controlled release formulations of ropinirole to reduce the dosing frequency and to provide therapeutic concentrations throughout the day with reduced fluctuations in plasma concentration, for the treatment of Parkinson's disease and other disorders.

U.S. Patent Application Publication Nos. 2004/0247676 A1, 2007/0264336 A1, and 2003/0180359 A1 disclose controlled release pharmaceutical compositions of ropinirole for therapeutic use.

U.S. Patent Application Publication No. 2007/0059365 A1 describes controlled release formulations of ropinirole for oral administration for use in the treatment of diseases which can prevent or disturb sleep, particularly restless leg syndrome.

There remains a need for alternative controlled release pharmaceutical compositions comprising ropinirole, which are simple, and can be prepared by cost effective processes, in order to reduce the dosing frequency and thereby improve patient compliance.

### SUMMARY

The present invention relates to controlled release pharmaceutical compositions of ropinirole or pharmaceutically acceptable salts thereof for therapeutic purposes, and methods of preparing the same.

In an embodiment, the invention includes controlled release pharmaceutical compositions comprising ropinirole or pharmaceutically acceptable salts thereof, wherein said ropinirole or pharmaceutically acceptable salts thereof is embedded in a matrix comprising one or more polymers, and the composition is in monolithic form.

In an embodiment, the invention includes a controlled release pharmaceutical composition comprising ropinirole or pharmaceutically acceptable salts thereof, wherein said ropinirole or pharmaceutically acceptable salts thereof is embedded in a matrix comprising one or more polymers, and said composition is in multiparticulate form.

In another embodiment, the invention includes controlled release pharmaceutical compositions comprising ropinirole or pharmaceutically acceptable salts thereof and one or more polymers, wherein ropinirole or pharmaceutically acceptable salts thereof is present in reservoir form.

In another embodiment, the invention includes controlled release pharmaceutical compositions comprising ropinirole or pharmaceutically acceptable salts thereof having a particle size distribution with D₉₀ (at least about 90 volume percent of the particles) less than about 100 μm, or less than about 50 μm, or less than about 20 μm.

In another embodiment, the invention includes a controlled release multi-layer tablet, comprising: (a) at least one active layer comprising ropinirole or pharmaceutically acceptable salts thereof and one or more polymers comprising any of: (i) hydrophilic polymeric substances which swell, gel, and/or erode upon contact with aqueous liquids; (ii) lipophilic substances; or (iii) combinations of hydrophilic and lipophilic substances wherein the weight percent of hydrophilic polymeric substances to the total weight of said hydrophilic and lipophilic substances is greater than 87.5, or greater than about 91, or less than about 50, or less than about 33; and (b) one or more barrier layers containing one or more polymers.

In another embodiment, the invention includes controlled release multi-layer tablets, comprising: (a) one active layer comprising ropinirole or pharmaceutically acceptable salts thereof; (b) one or more barrier layers; wherein at least one layer comprises one or more polymeric substances that do not swell, do not gel, and do not erode upon contact with aqueous liquids.

In another embodiment, the invention includes methods of preparing pharmaceutical compositions of the present invention.

In further embodiment the invention includes methods of treating patients suffering from Parkinson's disease, restless leg syndrome, fibromyalgia and other such disorders, using pharmaceutical compositions of the present invention.

### DETAILED DESCRIPTION

The present invention relates to controlled release pharmaceutical compositions comprising ropinirole or pharmaceutically acceptable salts thereof for oral administration, and methods of preparing the same.

As used herein the term “ropinirole” includes the compound ropinirole, and pharmaceutically acceptable salts of ropinirole, including polymorphs, solvates and hydrates thereof.

As used herein the term “pharmaceutically acceptable salts,” or “salts,” include acid addition salts prepared from inorganic or organic acids, including but not limited to maleic, fumaric, benzoic, ascorbic, pamoic, succinic, bismethylenesalicylic, methanesulfonic, ethanedisulfonic, acetic, oxalic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric, and nitric acids. The hydrochloric acid salt is most frequently used. Ropinirole hydrochloride is a white to pale greenish-yellow powder with a melting range of 243° C. to 250° C. and a solubility of 133 mg/mL in water.

As used herein the term “controlled release” means the release of the active substance, i.e., ropinirole, occurs from a pharmaceutical dosage form in a manner modified to occur at a time and/or a different rate than that from an immediate release product, such as a conventional swallowed tablet or capsule. Controlled release pharmaceutical compositions of the present invention release drug over periods of time at least about 12 hours, or at least about 16 hours, or at least about 20 hours, or at least about 24 hours, following administration. Sometimes the active substance may alternatively, or also, be present in “delayed release” form where the release of the active substance is modified to commence at a later time than that from an immediate release form. Optionally, controlled release pharmaceutical compositions of the present invention begin to release drug at least about 0.5 hours or at least about 1 hour, or at least about 2 hours following administration.

In an embodiment, the invention includes controlled release pharmaceutical compositions comprising ropinirole, wherein said ropinirole is embedded in a matrix comprising one or more polymers.

In another embodiment, the invention includes controlled release pharmaceutical compositions comprising ropinirole, wherein said ropinirole is embedded in a matrix comprising one or more polymers, and the composition is in monolithic form.

In another embodiment, the invention includes controlled release pharmaceutical compositions comprising ropinirole, one or more polymers and optionally other additives, wherein said ropinirole is uniformly distributed or embedded in a polymeric matrix, and the composition is in monolithic form.

A pharmaceutical composition according to the present invention can be presented in forms such as tablets, multilayered tablets, capsules, granules, spheroids, beads, pellets, minitablets, powders, sachets, gels, dispersions, solutions, or suspensions.

As used herein the term “particulate” means granules, spheroids, beads, pellets, or minitablets.

In an embodiment, the invention includes controlled release pharmaceutical compositions comprising about 0.05 to about 10% by weight of ropinirole, or about 0.1 to about 8% by weight of ropinirole, or about 0.5 to about 5% by weight of ropinirole.

In an embodiment, the invention includes controlled release pharmaceutical compositions comprising about 20 to about 95% by weight of one or more polymers, or about 35 to about 85% by weight of one or more polymers, or about 50 to about 75% by weight of one or more polymers.

In another embodiment, the invention includes controlled release pharmaceutical compositions comprising ropinirole, wherein the ropinirole is uniformly distributed or embedded in a matrix comprising one or more polymers, from which it is released by diffusion and/or erosion.

In one embodiment, the invention includes controlled release pharmaceutical compositions that are uncoated.

In another embodiment, pharmaceutical compositions of the invention optionally have one or more coatings which are functional or non-functional. Functional coatings include controlled release coatings; and non-functional coatings include seal coatings and elegant coatings.

In another embodiment, the pharmaceutical compositions are optionally coated with a composition comprising ropinirole.

In an embodiment, the invention includes controlled release pharmaceutical compositions comprising ropinirole, wherein said ropinirole is embedded in a matrix comprising one or more polymers, and said compositions are in multi particulate form.

In another embodiment, the invention includes controlled release pharmaceutical compositions comprising controlled release multiparticulates, wherein each particle comprises ropinirole and one or more polymers.

In one embodiment, the controlled release multiparticulates comprising ropinirole of the invention are composed of a core comprising ropinirole and one or more polymers, and optionally other additives.

In one embodiment, the multiparticulates comprising ropinirole are uncoated. In other embodiments, the multiparticulates comprising ropinirole comprise one or more coatings over a core.

In still other embodiments, the multiparticulates comprising ropinirole have a non-functional seal coating, a functional coating, or both.

In one embodiment, an initial seal coating can be applied directly to a core.

Optionally, the multiparticulates can contain a further coating layer comprising one or more polymers over an initial seal coating, if present, or directly over an uncoated multiparticulate ropinirole core, to provide further control of drug release.

In one embodiment, an enteric coating may be applied to ropinirole-containing multiparticulates.

In one embodiment, an enteric-coated multiparticulate is further coated with a final seal coating.

In still other embodiments, any one or all of the coating compositions optionally contain ropinirole.

In another embodiment, the invention includes controlled release pharmaceutical compositions comprising ropinirole and one or more polymers; wherein said ropinirole is present in reservoir form.

In another embodiment, the invention includes controlled release pharmaceutical compositions comprising a core comprising ropinirole and a coating comprising one or more polymers, and said compositions are in monolithic form.

In another embodiment, the invention includes controlled release pharmaceutical compositions comprising a core comprising a pharmaceutically acceptable additive, and a coating comprising ropinirole, and said compositions are in monolithic form.

In one embodiment, the invention includes controlled release pharmaceutical compositions optionally having one or more coatings which are functional or non-functional. Functional coatings include controlled release coatings, and non-functional coatings includes seal coatings and elegant coatings.

In another embodiment, the invention includes controlled release pharmaceutical compositions optionally having a coating comprising ropinirole.

The compositions of the present invention may be prepared using methods including wet granulation, melt granulation, dry granulation such as slugging or compaction, direct compression, and various coating processes, and are formulated into dosage forms including tablets and capsules.

In an embodiment, the invention includes controlled release pharmaceutical compositions comprising ropinirole and one or more polymers, wherein ropinirole is present in reservoir form and the composition is in multiparticulate form.

In another embodiment, the invention includes controlled release pharmaceutical compositions comprising a core comprising ropinirole and a coating comprising one or more polymers, and said compositions are in multiparticulate form.

In one embodiment, controlled release multiparticulates comprising ropinirole comprise cores comprising ropinirole and optionally one or more pharmaceutically acceptable additives, and a coating comprising one or more polymers.

In one embodiment, the controlled release multiparticulates comprising ropinirole comprise a non-panel core such as inert sugar or similar substances, upon which ropinirole is coated optionally along with other pharmaceutically acceptable additives, using any technique such as powder layering, solution spraying, or suspension spra'ying.

In one embodiment, the controlled release compositions of the invention are composed of ropinirole-loaded non-pariel cores, and a coating comprising one or more polymers.

In another embodiment, the invention includes pharmaceutical compositions comprising controlled release multiparticulates comprising ropinirole in a drug-containing core and a coating comprising one or more polymers, and optionally having one or more further coatings.

In still other embodiments, the multiparticulates comprising ropinirole contain a further coating, a non-functional seal coating or a functional coating or both.

In one embodiment, an initial seal coating can be applied directly to drug-containing cores.

In one embodiment, an enteric coating may be applied to the multiparticulates comprising ropinirole.

In one embodiment, enteric-coated multiparticulates are further coated with a final seal coating.

In still other embodiments, any one or all of the coating compositions optionally contain ropinirole.

The multiparticulate formulations of the invention can be prepared using techniques described herein, as well as methods known to those having skill in the art.

In another embodiment, portions of multiparticulates comprising ropinirole are coated with different concentrations of polymers, which will give different drug release profiles, and can be combined to form a pharmaceutical composition or dosage form to achieve desired controlled release.

In another embodiment, multiparticulates comprising ropinirole can be combined with a pharmaceutically acceptable carrier and optionally other additives and compounded to form a pharmaceutical composition, i.e., can be compressed into tablets or placed into suitable capsule shells, using techniques known to those having skill in the art.

In another embodiment, the invention includes controlled release pharmaceutical compositions comprising ropinirole and one or more lipids, wherein said ropinirole is entrapped in lipid vesicles.

In still other embodiments, the invention includes controlled release pharmaceutical compositions comprising ropinirole, one or more lipids, and optionally one or more emulsifiers and wetting agents or surfactants.

In another embodiment, the invention includes controlled release multi-layer tablets, comprising: (a) at least one active layer comprising ropinirole or pharmaceutically acceptable salts thereof and one or more polymers comprising any of: (i) hydrophilic polymeric substances that swell, gel, and/or erode upon contact with aqueous liquids; (ii) lipophilic substances; or (iii) combinations of hydrophilic and lipophilic substances wherein the weight percentage of hydrophilic polymeric substance to the total weight of said hydrophilic and lipophilic substances is greater than 87.5, greater than about 91, less than about 50, or less than about 33; and (b) one or more barrier layers containing one or more polymers.

The layers containing the active ingredient can be referred to as the “active layer” and the layer that generally does not contain an active substance can be referred to as a “barrier layer” or a “support layer.” Multi-layer tablets of the present invention can be prepared as two-layer tablets, three-layer tablets or in greater numbers of layers if desired. At least one layer will contain the active substance to be released from the tablet and least one layer will be a barrier or support layer with respect to the active substance-containing layer.

In one embodiment, the invention includes controlled release multi-layer tablets, comprising: (a) one active layer comprising ropinirole or pharmaceutically acceptable salts thereof, one or more hydrophilic polymeric substances which swell, gel, and/or erode upon contact with aqueous liquids, and one or more lipophilic substances, wherein the weight percentage of hydrophilic polymeric substances to the total weight of said hydrophilic and lipophilic substances is greater than 87.5 or greater than about 91; and (b) one or more barrier layers comprising one or more polymers.

In one embodiment, the invention includes controlled release multi-layer tablets, comprising: (a) one active layer comprising ropinirole or pharmaceutically acceptable salts thereof, one or more hydrophilic polymeric substances which swell, gel, and/or erode upon contact with aqueous liquids, and one or more lipophilic substances, wherein the weight percentage of hydrophilic polymeric substances to the total weight of said hydrophilic and lipophilic substances is less than about 50, or less than about 33; and (b) one or more barrier layers comprising one or more polymers.

In another embodiment, the invention includes controlled release multi-layer tablets, comprising: (a) one active layer comprising ropinirole or pharmaceutically acceptable salts thereof; and (b) one or more barrier layers; wherein the multi-layer tablets comprise at least one layer comprising one or more polymeric substances that do not swell, do not gel, and do not erode upon contact with aqueous liquids.

In one embodiment, the invention includes controlled release multi-layer tablets, comprising: (a) one active layer comprising ropinirole or pharmaceutically acceptable salts thereof and one or more polymeric substances which do not swell, do not gel, and do not erode upon contact with aqueous liquids; and (b) one or more barrier layers comprising one or more polymers.

In one embodiment, the invention includes controlled release multi-layer tablets, comprising: (a) one active layer comprising ropinirole or pharmaceutically acceptable salts thereof and one or more polymers; and (b) one or more barrier layers comprising one or more polymeric substances which do not swell, do not gel, and do not erode upon contact with aqueous liquids.

In one embodiment, the invention includes controlled release multi-layer tablets, comprising: (a) one active layer comprising ropinirole or pharmaceutically acceptable salts thereof, one or more polymers and one or more adjuvant substances, wherein the weight percentage of one or more adjuvant substances to the total weight of said active layer is more than about 50; and (b) one or more barrier layers comprising one or more polymers.

In one embodiment, the invention includes controlled release multi-layer tablets, comprising: (a) one active layer comprising ropinirole or pharmaceutically acceptable salts thereof and one or more adjuvant substances; and (b) one or more barrier layers comprising one or more polymers.

In still other embodiments, the invention includes controlled release multi-layer tablets, wherein the barrier layer may also contain an active substance that acts as a barrier layer with respect to a first active substance-containing layer, and which itself is an active substance-containing layer.

In one embodiment, the invention includes controlled release multi-layer tablets that are uncoated.

In another embodiment, the invention includes controlled release multi-layer tablets optionally having one or more coatings which are functional or non-functional. Functional coatings include controlled release coatings, and non-functional coatings include seal coatings and elegant coatings.

In another embodiment, a multi-layer tablet is optionally coated with a composition comprising ropinirole.

Multi-layer tablets of the invention can be prepared using methods known to those having skill in the art.

In an embodiment, the invention includes controlled release osmotic devices comprising: (a) a core comprising ropinirole or a pharmaceutically acceptable salt thereof, an osmotic agent and optionally other pharmaceutically acceptable additives; (b) a semipermeable wall surrounding the core; and (c) at least one passageway through the wall.

The osmotic agents that can be used for the purpose of this invention include inorganic and organic compounds that exhibit an osmotic pressure gradient across a semipermeable wall against an external fluid. Osmotic agents useful in the present invention include, without limitation thereto, sodium chloride, magnesium sulfate, magnesium chloride, potassium sulfate, sodium sulfate, lithium sulfate, potassium acid phosphate, d-mannitol, urea, inositol, magnesium succinate, tartaric acid, carbohydrates such as raffinose, sucrose, glucose, and mixtures thereof.

The semipermeable wall is permeable to the passage of an external fluid such as water and biological fluids, and is substantially impermeable to the passage of active agents, osmotic agents, and the like. Typical materials for forming the wall are in some embodiments cellulose esters, cellulose ethers and cellulose ester-ethers. Representative materials include, without limitation thereto, cellulose acylate, cellulose diacylate, cellulose triacylate, cellulose acetate, cellulose diacetate, cellulose triacetate, moni, di and tricellulose alkanylates, mono, di and tricellulose aroylates, and the like.

The passageway as used herein comprises means and methods suitable for releasing the agent or drug from the osmotic system. The passageway includes an osmotic aperture or osmotic orifice through the semipermeable wall. The osmotic passageway can be formed by techniques including mechanical drilling or laser drilling.

The controlled-release ropinirole compositions of the present invention are advantageous as they establish more constant plasma concentrations with reduced fluctuations over a prolonged period of time, when compared to an immediate-release composition, and avoid the necessity for the immediate-release composition of taking ropinirole three times each day. This tends to alleviate some of the side-effects which are observed when immediate release ropinirole is administered.

In another embodiment, the invention includes controlled release oral dosage forms comprising ropinirole, characterized by mean times to achieve a half-peak plasma concentration (0.5 Cₘₐₓ) of ropinirole in vivo more than 3 hours after administration of the oral dosage form.

In another embodiment, the invention includes controlled release pharmaceutical compositions comprising ropinirole, wherein said compositions release at least about 10% by weight of the contained ropinirole into a lower part of the gastrointestinal tract.

By “lower part of gastrointestinal tract” is meant the portion of the gastrointestinal tract between the region of the ileo-caecal junction and the rectum, inclusive.

The different physicochemical properties of the active ingredient and as well as of excipients are to be considered, as these properties affect the process and formulation properties of the compound. Various important physicochemical properties include but are not limited to particle sizes and distributions of the active ingredient, density (bulk density and tapped density), compressibility index, Hausner's ratio, angle of repose, etc. These physicochemical properties not only affect the process of the preparing the pharmaceutical compositions but also affect the performance of the pharmaceutical product, both in vitro and in vivo.

Particle sizes of active pharmaceutical ingredient (API) can affect the solid dosage form in numerous ways. For example, content uniformity (CU) of pharmaceutical dosage units can be affected by particle size and size distribution.

This will be even more critical for low-dose drugs like ropinirole. Satisfactory dosage units of low doses cannot be manufactured from a drug that does not meet certain particle size and size distribution criteria.

Ropinirole as an active ingredient poses some problems, while processing it into pharmaceutical compositions, particularly into tablets. It will give stickiness during compression, due to having an electrical charge, and leads to various process related problems such as CU variation, which can make processes unacceptable.

In an embodiment, the invention includes controlled release pharmaceutical compositions comprising ropinirole and one or more polymers, wherein ropinirole has a particle size distribution with D₉₀ (at least 90 volume percent of the particles) less than about 100 μm, or less than about 50 μm, or less than about 20 pin.

The D₁₀, D₅₀, and D₉₀ values are useful ways for indicating a particle size distribution. D₉₀ is the size value where at least 90 volume percent of the particles have a size smaller than the said value. Likewise D₁₀ refers to 10 volume percent of the particles having a size smaller than the said value. D₅₀ refers to at least 50 volume percent of the particles having a size smaller than the said value and a D_{[4,3}] value refers to the mean particle size. Methods for determining D₁₀, D₅₀ D₉₀ and D_{[4,3]} include laser diffraction, such as using equipment sold by Malvem Instruments Ltd., Malvern, Worcestershire, United Kingdom.

In another embodiment, the invention includes pharmaceutical compositions comprising ropinirole and one or more pharmaceutical aids to enhance processability.

One or more pharmaceutical aids that can be used in the present invention include adsorbents. Suitable adsorbents include, but are not limited to, colloidal silicon dioxide, calcium silicate, other silicates, kaolin, microcrystalline cellulose, bentonite, talc, sorbitol, clays, Fujicalin™, Neusilin™, Zeopharm™, magnesium carbonate, magnesium oxide, calcium carbonate, resins, crospovidone, sugar, vinegar and diatomaceous earth materials, lactose, etc. used singly or in mixtures.

One or more polymers that can be used in the present invention include hydrophilic, hydrophobic and lipophilic substances, and combinations thereof. Examples of polymers which swell, and/or gel, and/or erode upon contact with aqueous liquids, that can be used in present invention include, without limitation thereto, cellulose ethers, e.g., hydroxypropyl methylcelluloses or hypromelloses (HPMC), hydroxypropylcelluloses (HPC), hydroxyethylcelluloses, ethylcelluloses and carboxymethylcellulose sodium, polymers of vinylpyrrolidone, including homopolymers (povidones) and cross-linked polyvinylpyrrolidones, carboxymethylstarches, polyethylene glycols, polyoxyethylenes, poloxamers (polyoxyethylene-polyoxypropylene copolymers), polyvinylalcohols, glucanes (glucans), carrageenans, scleroglucanes (scleroglucans), mannans, galactomannans, gellans, alginic acid and derivatives (e.g., sodium or calcium alginate, propylene glycol alginate), polyaminoacids (e.g. gelatin), methyl vinyl ether/maleic anhydride copolymers, polysaccharides (e.g. carageenan, guar gum, xanthan gum, tragacanth and ceratonia), alpha, beta or gamma cyclodextrins, and dextrin derivatives (e.g. dextrin), polymethacrylates (e.g. copolymers of acrylic and methacrylic acid esters containing quaternary ammonium groups); acrylic acid polymers (e.g., carbomers); shellac, and derivatives thereof, etc.

Examples of polymers, which do not swell and do not gel and do not erode upon contact with aqueous liquids, that can be used in the present invention include, without limitation thereto, cellulose acetate, cellulose butyrate, cellulose diacetate, cellulose triacetate, cellulose propionate, cellulose acetate butyrate, and other acetylated cellulose derivatives, etc.

Examples of lipophilic substances that can be used in the present invention include, without limitation thereto, waxes (e.g., carnauba wax, microcrystalline wax, beeswax, polyethoxylated beeswax); natural fats (coconut, soya, cocoa) including modified forms such as totally or partially hydrogenated, hydrogenated castor oil, hydrogenated vegetable oil, and fatty acid derivatives such as mono-, bi- and tri-substituted glycerides, phospholipids, glycerophospholipids, glyceryl palmitostearate, glyceryl behenate, glyceryl monostearate, diethyleneglycol palmitostearate, polyethyleneglycol stearate, polyethyleneglycol palmitostearate, polyoxyethylene-glycol palmitostearate, glyceryl monopalmitostearate, cetyl palmitate, fatty alcohols associated with polyethoxylate fatty alcohols, cetyl alcohol, stearic acid, saturated or unsaturated fatty acids and their hydrogenated derivatives, lecithin, cephalins, chitosan and derivatives thereof, sphingolipids, sterols such as cholesterol and its substituted derivatives, etc.

In certain embodiments, pharmaceutical compositions of the present invention optionally include additives additional to the active agent and one or more polymers, which includes without limitation diluents, binders, disintegrants, surfactants, and other additives that are commonly used in solid dosage form preparations.

### Diluents:

Various useful fillers or diluents include but are not limited to starches, lactose, mannitol (Pearlitol™ SD200), cellulose derivatives, confectioner's sugar and the like. Different grades of lactose include but are not limited to lactose monohydrate, lactose DT (direct tableting), lactose anhydrous, Flowlac™ (available from Meggle Products), Pharmatose™ (available from DMV) and others. Different starches include but are not limited to maize starch, potato starch, rice starch, wheat starch, pregelatinized starch (commercially available as PCS PC10 from Signet Chemical, Corporation) and starch 1500, starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, PH-112 microcrystalline cellulose 114, and microcrystalline cellulose 112. Other useful diluents include but are not limited to carmellose, sugar alcohols such as mannitol (Pearlitol™ SD200), sorbitol and xylitol, calcium carbonate, magnesium carbonate, dibasic calcium phosphate, and tribasic calcium phosphate.

### Binders:

Various useful binders include but are not limited to hydroxypropylcelluloses, also called HPC (Klucel™-LF, Klucel EXF) and useful in various grades, hydroxypropyl methylcelluloses; also called hypromelloses or HPMC (Methocel™) and useful in various grades, polyvinylpyrrolidones or povidones (such as grades PVP-K25, PVP-K29, PVP-K30, and PVP-K90), Plasdone™ S 630 (copovidone), powdered acacia, gelatin, guar gum, carbomers (Carbopol™), methylcelluloses, polymethacrylates, and starches.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcelluloses include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Surface-Active Agents:

Useful surface-active agents include non-ionic, cationic and anionic surface-active agents. Useful non-ionic surface-active agents include ethylene glycol stearates, propylene glycol stearates, diethylene glycol stearates, glycerol stearates, sorbitan esters (SPAN™) and polyhydroxyethylenically treated sorbitan esters (TWEEN™), aliphatic alcohols and PEG ethers, phenol and PEG ethers. Useful cationic surface-active agents include quaternary ammonium salts (e.g., cetyltrimethylammonium bromide) and amine salts (e.g., octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidyl glycerols, diaceylphosphatidyl cholines, and diaceylphosphatidic acids, the precursors and derivatives thereof, such as for example soybean lecithin and egg yolk.

### Lubricants:

An effective amount of any pharmaceutically acceptable tableting lubricant can be added to assist with compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Glidants:

One or more glidant materials, which improve the flow properties of powder blends and minimize dosage form weight variation can be used. Useful glidants include but are not limited to silicone dioxide, talc and combinations thereof.

### Coloring Agents:

Coloring agents can be used to color code the compositions, for example, to indicate the type and dosage of the therapeutic agent therein. Suitable coloring agents include, without limitation, natural and/or artificial compounds such as FD&C coloring agents, natural juice concentrates, pigments such as titanium oxide, iron oxides, silicon dioxide, zinc oxide, combinations thereof, and the like.

### Solvents:

Various solvents can be used in the processes for preparation of pharmaceutical compositions of the present invention, including but not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, dimethylformamide, tetrahydrofuran and mixtures thereof.

Coating materials used for film coating include the polymers described above. Enteric coating materials include but are not limited to materials such as cellulosic polymers like cellulose acetate phthalates, cellulose acetate trimellitates, hydroxypropyl methylcellulose phthalates, polyvinyl acetate phthalates, etc., methacrylic acid polymers and copolymers (Eudragit™), and the like, and mixtures thereof.

Other useful additives for coating include but are not limited to plasticizers, antiadherents, opacifiers, solvents, and optionally colorants, lubricants, pigments, antifoam agents, and polishing agents.

Various useful plasticizers include but are not limited to substances such as castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent. An opacifier like titianium dioxide may also be present in an amount ranging from about 10% (w/w) to about 20% (w/w) based on the total weight of the coating.

Antiadhesives are frequently used in the film coating process to avoid sticking effects during film formation and drying. An example of a useful antiadhesive for this purpose is talc. The antiadhesive is frequently present in the film coating in an amount of about 5% (w/w) to about 15% (w/w) based upon the total weight of the coating.

When coloured tablets are desired, the colour is normally applied in the coating. Consequently, colouring agents and pigments may be present in the film coating. Various colouring agents include but not limited to iron oxides, which can be red, yellow, black or blends thereof.

Suitable polishing agents include polyethylene glycols of differing molecular weights and mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In some embodiments, polyethylene glycols having molecular weights of 3,000-20,000 are employed.

As alternatives to the above coating ingredients, sometimes pre-formulated coating products such as OPADRY™ (supplied by Colorcon) will be used. Opadry compositions generally comprise polymer, plasticizer and, if desired, pigment in a dry concentrate that requires only dispersion in a liquid prior to use. Opadry formulas produce attractive, elegant coatings on a variety of tablet cores and can be used in both aqueous and organic coating procedures.

In an embodiments, the invention includes methods for preparing pharmaceutical compositions of the present invention.

In one aspect the invention includes processes for preparing pharmaceutical compositions, an embodiment of a process comprising:

i) sifting ropinirole, a polymer and optionally a diluent through a suitable sieve and mixing;

ii) making a binder solution by dissolving a binder in suitable solvent;

iii) granulating the mixture of step i) using the binder solution of step ii);

iv) drying the granules formed in step iii);

v) blending the dried granules with optional extragranular additives such as a glidant; and

vi) blending the mixture of v) with a lubricant, followed by compressing into tablets.

In another aspect the invention includes processes for preparing pharmaceutical compositions, an embodiment of a process comprising:

i) sifting ropinirole, polymer, diluent and optionally other additives through a suitable sieve and mixing;

ii) blending the mixture of step i) with a lubricant, followed by compressing into tablets; and

iii) optionally, coating tablets of step ii) with a suitable enteric polymer.

In another aspect the invention includes processes for preparing pharmaceutical compositions, an embodiment of a process comprising:

i) dissolving ropinirole and a binder in a suitable solvent;

ii) coating non-pareil seeds with drug solution of step i) in a fluid bed coating apparatus with intermittent drying;

iii) making a controlled release coating composition by dissolving one or more polymers, a plasticizer and other additives in a suitable solvent;

iv) coating drug loaded beads of step ii) with coating solution of step iii) in a fluid bed coating apparatus with intermittent drying;

v) optionally, coating controlled release beads of step iv) with a suitable enteric polymer;

vi) mixing the beads of step iv) or v) with optional additives such as a glidant; and

vii) blending the mixture of step vi) with a lubricant, followed by compressing into tablets.

In another aspect the invention includes processes for preparing pharmaceutical compositions, an embodiment of a process comprising:

i) sifting ropinirole, polymer and optionally diluent and other additives through a suitable sieve and mixing;

ii) making a binder solution by dissolving a binder in a suitable solvent and adding to the mix of step i) to make a wet mass;

iii) preparing controlled release beads by an extrusion-spheronization technique using the wet mass of step ii);

iv) optionally, coating controlled release beads of step (iii) with a controlled release coating composition prepared by dissolving one or more polymers, a plasticizer and other additives in suitable solvent;

v) optionally coating controlled release beads of step iii) or iv) with suitable enteric polymers using a suitable solvent system;

vi) blending the beads of step iii), iv) or v) with optional additives such as a glidant; vii) blending the mixture of step vi) with a lubricant, followed by compressing into tablets.

Dosage forms can be subjected to in vitro dissolution evaluation, such as according to Test 711 “Dissolution” in United States Pharmacopoeia 29, United States Pharmacopeial Convention, Inc., Rockville, Md., 2005 (“USP”) to determine the rate at which the active substance is released from the dosage forms, and content of active substance can be determined in dissolution solutions by techniques such as high performance liquid chromatography.

In an embodiment, the invention includes controlled release monolithic tablets or multiparticulate pharmaceutical dosage forms containing ropinirole, from which ropinirole completely dissolves over a period of more than 12 hours following immersion in an aqueous fluid.

In another embodiment, the invention includes controlled release monolithic tablets or multiparticulate pharmaceutical dosage forms containing ropinirole, from which ropinirole completely dissolves over a period of more than 16 hours following immersion in an aqueous fluid.

In another embodiment, the invention includes controlled release monolithic tablets or multiparticulate pharmaceutical dosage forms containing ropinirole, from which ropinirole completely dissolves over a period of more than 20 hours following immersion in an aqueous fluid.

In another embodiment, the invention includes controlled release monolithic tablets or multiparticulate pharmaceutical dosage forms containing ropinirole, from which ropinirole completely dissolves over a period of more than 24 hours following immersion in an aqueous fluid.

As used herein the term “complete dissolution” means release of at least about 80 percent of the labeled dose of ropinirole when measured by the USP Paddle method (USP Apparatus II) at 100 rpm in 500 ml of aqueous buffer (physiological pH range between 1 and 7) at 37° C.

In one embodiment, the invention includes controlled release monolithic tablets or multiparticulate pharmaceutical dosage forms containing ropinirole, wherein not more than about 30 percent of ropinirole dissolves within about 1 hour following immersion in an aqueous fluid.

In one embodiment, the invention includes controlled release monolithic tablets or multiparticulate pharmaceutical dosage forms containing ropinirole, wherein about 40 percent to about 70 percent of ropinirole dissolves within about 8 hours to 10 hours following immersion in an aqueous fluid.

In an embodiment, the invention includes controlled release monolithic tablets or multiparticulate pharmaceutical dosage forms containing ropinirole, wherein about 50 percent to about 80 percent of ropinirole dissolves within about 12 hours to 14 hours following immersion in an aqueous fluid.

In another embodiment, the invention includes controlled release monolithic tablets or multiparticulate pharmaceutical dosage forms containing ropinirole, wherein about 70 percent to about 90 percent of ropinirole dissolves within about 16 hours to 20 hours following immersion in an aqueous fluid.

When comparing the test and reference products, dissolution profiles can be compared using a similarity factor (f₂). The similarity factor is a logarithmic reciprocal square root transformation of the sum of squared error, and is a measurement of the similarity in the percentages of dissolution between the two curves. It is determined through a point-by-point comparison of a new composition's in vitro dissolution profile with a reference composition's in vitro dissolution profile, as shown in the following equation:

f₂=50×log {[1+(1/n)Σₜ₌₁ⁿ(Rₜ−Tₜ)²]^{-0.5}×100}.

Rₜ refers to the percent of compound dissolved at each time point (t) for the reference. Tₜ refers to the percent of compound dissolved at each time point (t) for the test sample, n refers to the number of time points used for the calculation. Two dissolution profiles are considered similar when the f₂ value is equal to or greater than 50. (“Guidance for Industry, Waiver of In Vivo Bioavailability and Bioequivalence Studies for Immediate-Release Solid Oral Dosage Forms Based on a Biopharmaceutics Classification System,” U.S. Department of Health and Human Services, Food and Drug Administration, Center for Drug Evaluation and Research, August 2000, page 8)

In one of the embodiments, the present invention provides controlled release pharmaceutical compositions of ropinirole with specific dissolution profiles. For the purpose of this invention, in vitro dissolution rates or profiles of the active ingredient of the pharmaceutical composition are measured in the pharmaceutical composition according to the conditions in Example 25.

In one embodiment, the invention includes controlled release pharmaceutical compositions comprising ropinirole or a pharmaceutically acceptable salt thereof as an active ingredient, wherein the entire active ingredient has an in vitro dissolution profile with a similarity factor (f₂) of at least 50 to 100, compared to a reference composition dissolution profile as shown in Example 25.

In one embodiment, the invention includes controlled release pharmaceutical compositions comprising ropinirole or a pharmaceutically acceptable salt thereof as an active ingredient and one or more polymers, wherein said composition is in monolithic form and the entire active ingredient has an in vitro dissolution profile with a similarity factor (f₂) of at least 50 to 100, compared to a reference dissolution profile as shown in Example 25.

In one embodiment, the invention includes controlled release pharmaceutical compositions comprising ropinirole or a pharmaceutically acceptable salt thereof as an active ingredient and one or more polymers, wherein said composition is in multiparticulate form and the entire active ingredient has an in vitro dissolution profile with a similarity factor (f₂) of at least 50 to 100, compared to a reference dissolution profile as shown in Example 25.

In an embodiment, f₂ is about 60 to 100. In an embodiment, f₂ is about 65 to 100. In another embodiment, f₂ is about 80 to 100.

In embodiments, the invention includes controlled release pharmaceutical compositions comprising ropinirole or a pharmaceutically acceptable salt thereof as an active ingredient, wherein the compositions are bioequivalent to the commercial product Requip® XL, which is a trilayer tablet.

“Bioequivalence” of two pharmaceutical products can be determined by administering the products individually to a number of subjects, and determining blood levels of a contained drug substance at intervals thereafter. The products are considered to be bioequivalent if one or more calculated bioavailability pharmacokinetic parameters are similar, i.e., have a relationship where administration of a “test” product produces pharmacokinetic parameters that are within the range of 80% to 125% of the values obtained from administering a “reference” product. Frequently used pharmacokinetic parameters for indicating bioequivalence include:

AUC₀₋ₜ=Area under a plasma drug concentration versus time curve, from time zero (administration) to the last measurable concentration.

AUC_{0-∞}=Area under a plasma drug concentration versus time curve, from time zero (administration) to infinity.

Cₘₐₓ=Maximum plasma drug concentration.

Tₘₐₓ=Elapsed time from administration until the maximum measured plasma drug concentration.

In an embodiment, the invention includes controlled release pharmaceutical compositions comprising ropinirole or a pharmaceutically acceptable salt thereof and one or more polymers, wherein the compositions are in monolithic form and are bioequivalent to a reference commercial product, which is a trilayer tablet.

In another embodiment, the invention includes controlled release pharmaceutical compositions comprising ropinirole or a pharmaceutically acceptable salt thereof and one or more polymers, wherein the compositions are in multiparticulate form and are bioequivalent to a reference commercial product, which is a trilayer tablet.

In some embodiments, the invention includes use of packaging materials such as containers and lids of high-density polyethylene (HDPE), low-density polyethylene (LDPE) and or polypropylene and/or glass, and blisters or strips composed of aluminium or high-density polypropylene, polyvinyl chloride, polyvinylidene dichloride, etc.

Embodiments of the present invention provide unit doses of ropinirole about 0.75 to about 12 milligrams per dosage form.

In further embodiments the invention includes methods for treating patients suffering from Parkinson's disease, restless leg syndrome, fibromyalgia and other such disorders, using pharmaceutical compositions of the present invention.

The pharmaceutical dosage forms of the present invention are intended for oral administration to a patient in need thereof.

Certain specific aspects and embodiments of the invention will be explained in more detail with reference to the following examples, being provided only for purposes of illustration, and it is to be understood that the present invention is not deemed to be limited thereto.

### Example 1

### Ropinirole 8 mg Controlled Release Tablets

| | mg/Tablet | |
|---|---|---|
| Ingredient | Example 1A | Example 1B |
| Ropinirole hydrochloride** | 9.12 | 9.12 |
| HPMC K 100M | 180 | 185 |
| HPMC K 15M | 60 | 55 |
| Povidone K-30 | 15 | 15 |
| Microcrystalline cellulose PH 102 | 33.38 | 33.38 |
| Colloidal silicon dioxide | 1 | 1 |
| Magnesium stearate | 1.5 | 1.5 |
| Total | 300 | 300 |

**Equals 8 mg of ropinirole, particle size distribution of ropinirole hydrochloride D₉₀ < 13 μm.

Manufacturing process:

1. Mixed ropinirole hydrochloride geometrically with HPMC K 100M, HPMC K15M, povidone K-30, microcrystalline cellulose and colloidal silicon dioxide, and sifted through an ASTM #40 mesh sieve three times.

2. Sifted magnesium stearate through an ASTM # 60 mesh sieve.

3. Lubricated the material from step 1 by blending with the material of step 2.

4. Compressed the lubricated blend into tablets.

### Example 2

### Ropinirole 8 mg Controlled Release Tablets

| Ingredient | mg/Tablet |
|---|---|
| Ropinirole hydrochloride | 9.12 |
| HPMC K 100M | 100 |
| Sodium alginate | 25 |
| Hydroxypropylcellulose (HPC) HF | 15 |
| Ethylcellulose | 50 |
| Mannitol SD 200 | 98.38 |
| Water* | 0.533 |
| Colloidal silicon dioxide | 1 |
| Magnesium stearate | 1.5 |
| Total | 300 |

*Evaporates during processing.

Manufacturing process:

1. Mixed ropinirole hydrochloride geometrically with HPMC K 100M, sodium alginate, HPC—HF, and mannitol SD 200, and sifted twice through an ASTM #40 mesh sieve.

2. Granulated the material from step 1 using water, followed by drying.

3. Mixed ethylcellulose and colloidal silicon dioxide and sifted through an ASTM #40 mesh sieve.

4. Added the material from step 3 to the dried granules of step 2 and blended.

5. Sifted magnesium stearate through an ASTM #60 mesh sieve.

6. Lubricated the contents from step 4 by blending with magnesium stearate of step 5.

7. Compressed the lubricated blend into tablets.

### Example 3

### Ropinirole 8 mg Controlled Release Tablets

| | mg/Tablet | |
|---|---|---|
| Ingredient | Example 3A | Example 3B |
| Ropinirole hydrochloride | 9.12 | 9.12 |
| Hydrogenated castor oil | 45 | 105 |
| Stearic acid | 25 | 26 |
| Microcrystalline cellulose PH 102 | 50 | 50 |
| Povidone K-30 | 15 | — |
| Lactose DCL -21 | 151.88 | 107.38 |
| Colloidal silicon dioxide | 2.5 | 1 |
| Talc | 1.5 | 1.5 |
| Total | 300 | 300 |

Manufacturing process:

1. Melted hydrogenated castor oil and stearic acid and dispersed ropinirole hydrochloride uniformly into it.

2. Adsorbed the molten mass of step 1 onto a mixture of microcrystalline cellulose and colloidal silicon dioxide, previously sifted through an ASTM #40 mesh sieve, and screened through an ASTM #20 mesh sieve.

3. Added lactose and povidone (only for Example 3A) to the above and blended.

4. Sifted talc through an ASTM #60 mesh sieve.

5. Lubricated the blend of step 3 by mixing with talc of step 4.

6. Compressed the lubricated blend into tablets.

### Example 4

### Ropinirole 8 mg Controlled Release Tablets

| Ingredient | mg/Tablet |
|---|---|
| Ropinirole hydrochloride | 9.12 |
| HPMC K 100M | 50 |
| Hydrogenated castor oil | 25 |
| Povidone K-30 | 15 |
| Lactose | 198.38 |
| Colloidal silicon dioxide | 1 |
| Magnesium stearate | 1.5 |
| Total | 300 |

Manufacturing process:

1. Mixed ropinirole hydrochloride geometrically with HPMC K 100M, Hydrogenated castor oil, povidone K-30, lactose and colloidal silicon dioxide, and sifted through an ASTM #40 mesh sieve three times.

2. Sifted magnesium stearate through an ASTM # 60 mesh sieve.

3. Lubricated the material from step 1 by blending with the material of step 2.

4. Compressed the lubricated blend into tablets.

### Examples 5-6

### Ropinirole 8 mg Controlled Release Tablets

| | mg/Tablet | |
|---|---|---|
| Ingredient | Example 5 | Example 6 |
| Ropinirole hydrochloride | 9.12 | 9.12 |
| Ethylcellulose 7cps | 60 | 100 |
| Cellulose acetate | — | 50 |
| Mannitol | 60 | — |
| Lactose DCL 21 | — | 73.38 |
| Microcrystalline cellulose PH 102 | 168.38 | 50 |
| Povidone K 30 | — | 15 |
| Methanol* | 716 | 716 |
| Colloidal silicon dioxide | 1 | 1 |
| Talc | — | 0.75 |
| Magnesium stearate | 1.5 | 0.75 |
| Total | 300 | 300 |

*Evaporates during processing.

Example 5 manufacturing process:

1. Dissolved ropinirole hydrochloride and ethylcellulose in methanol, then dispersed mannitol in the solution.

2. Mixed microcrystalline cellulose with colloidal silicon dioxide in a polyethylene bag and sifted through an ASTM #40 mesh sieve.

3. Granulated the material of step 2 with the material from step 1 and dried.

4. Sifted the dried granules of step 3 through an ASTM #20 mesh sieve.

5. Sifted magnesium stearate through an ASTM #60 mesh sieve.

6. Lubricated the contents from step 4 by blending with magnesium stearate of step 5.

7. Compressed the lubricated blend into tablets.

Example 6 manufacturing process:

1. Dissolved ropinirole hydrochloride, cellulose acetate and ethylcellulose in methanol.

2. Mixed microcrystalline cellulose, lactose, povidone and colloidal silicon dioxide in a polyethylene bag and sifted through an ASTM #40 mesh sieve.

3. Granulated the material of step 2 using the solution of step 1 as a granulating agent and dried.

4. Sifted the dried granules of step 3 through an ASTM #20 mesh sieve.

5. Sifted talc through an ASTM #60 mesh sieve.

6. Added the sifted granules of step 4 to the talc of step 5.

7. Sifted magnesium stearate through an ASTM #60 mesh sieve.

8. Lubricated the contents from step 6 by blending with magnesium stearate of step 7 and compressed into tablets.

### Example 7

### Ropinirole 8 mg Controlled Release Tablets

| Ingredient | mg/Tablet |
|---|---|
| Ropinirole hydrochloride | 9.12 |
| HPMC K 100M | 125 |
| Hydrogenated castor oil | 75 |
| Stearic acid | 19 |
| Microcrystalline cellulose PH 102 | 50 |
| Lactose DCL 21 | 18.88 |
| Colloidal silicon dioxide | 1.5 |
| Talc | 1.5 |
| Total | 300 |

Manufacturing process:

1. Melted hydrogenated castor oil and stearic acid and dispersed ropinirole hydrochloride uniformly into it.

2. Adsorbed the molten mass of step 1 onto a mixture of HPMC K 100M, microcrystalline cellulose and colloidal silicon dioxide, previously sifted through an ASTM #40 mesh sieve, and allowed the mixture to solidify.

3. Sifted the solidified mixture of step 2 through an ASTM #20 mesh sieve.

4. Added lactose to the sifted mixture of step 3 and blended.

5. Sifted talc through an ASTM #60 mesh sieve.

6. Lubricated the blend of step 4 by mixing with talc of step 5 and compressed into tablets.

### Example 8

### Ropinirole 8 mg Controlled Release Tablets

| Ingredient | mg/Tablet |
|---|---|
| Ropinirole hydrochloride | 9.12 |
| HPMC K 100M | 140 |
| Sodium alginate | 40 |
| Hydroxypropylcellulose (HPC) 15 cps | 20 |
| Ethylcellulose 100 cp | 75 |
| Mannitol SD 200 | 13.38 |
| Methanol* | 716 |
| Colloidal silicon dioxide | 1 |
| Magnesium stearate | 1.5 |
| Total | 300 |

*Evaporates during processing.

Manufacturing process:

1. Dissolved ropinirole hydrochloride and ethylcellulose in methanol.

2. Mixed geometrically HPC with HPMC K 100M, sodium alginate and mannitol SD 200 and sifted twice through an ASTM #40 mesh sieve.

3. Granulated the material of step 2 using solution of step 1 as a granulating agent, followed by drying.

4. Sifted colloidal silicon dioxide through an ASTM #40 mesh sieve.

5. Added the material from step 4 to the dried granules of step 3 and blended.

6. Sifted magnesium stearate through an ASTM #60 mesh sieve.

7. Lubricated the contents from step 5 by blending with magnesium stearate of step 6.

8. Compressed the lubricated blend of step 7 into tablets.

### Examples 9-10

### Ropinirole 2 mg Controlled Release Tablets

| | mg/Tablet | |
|---|---|---|
| Ingredient | Example 9 | Example 10 |
| Ropinirole hydrochloride** | 2.28 | 2.28 |
| HPMC K 100M | 185 | — |
| HPMC K 15M | 55 | — |
| Hydrogenated castor oil | — | 96 |
| Stearic acid | — | 24 |
| Povidone K-30 | 15 | — |
| Microcrystalline cellulose PH 102 | 40.22 | 50 |
| Lactose DCL -21 | — | 125.22 |
| Colloidal silicon dioxide | 1 | 1 |
| Magnesium stearate | 1.5 | — |
| Talc | — | 1.5 |
| Total | 300 | 300 |

**Equals 2 mg of ropinirole.

Example 9 manufacturing process: similar to the process described in Example 1.

Example 10 manufacturing process: similar to the process described in Example 3.

### Example 11

### Ropinirole 8 mg Controlled Release Tablets

| Ingredient | mg/Tablet |
|---|---|
| Ropinirole hydrochloride** | 9.12 |
| HPMC K 100M | 184 |
| HPMC K 15M | 54 |
| Povidone K-30 | 10 |
| Sodium carboxymethylcellulose | 20 |
| Microcrystalline cellulose PH 102 | 20.38 |
| Colloidal silicon dioxide | 1 |
| Magnesium stearate | 1.5 |
| Total | 300 |

Manufacturing process:

1. Mixed ropinirole hydrochloride geometrically with HPMC K 100M, HPMC K15M, sodium carboxymethylcellulose, povidone K-30, microcrystalline cellulose and colloidal silicon dioxide, and sifted through an ASTM #40 mesh sieve three times.

2. Sifted magnesium stearate through an ASTM #60 mesh sieve.

3. Lubricated the material from step 1 by blending with the material of step 2, and compressed into tablets.

### Example 12

### Ropinirole 8 mg Controlled Release Tablets

| Ingredient | mg/Tablet |
|---|---|
| Ropinirole hydrochloride | 9.12 |
| HPMC K 100M | 50 |
| Hydrogenated castor oil | 25 |
| Povidone K 30 | 15 |
| Lactose | 198.38 |
| Colloidal silicon dioxide | 1 |
| Magnesium stearate | 1.5 |
| Total | 300 |

Manufacturing process:

1. Melted hydrogenated castor oil and dispersed ropinirole hydrochloride uniformly into it.

2. Adsorbed the molten mass of step 1 onto a mixture of HPMC K 100M, povidone and lactose: previously sifted through an ASTM #40 mesh sieve, and allowed the mixture to solidify.

3. Sifted the solidified mixture of step 2 through an ASTM #20 mesh sieve.

4. Sifted colloidal silicon dioxide through an ASTM #40 mesh sieve.

5. Added colloidal silicon dioxide of step 4 to the sifted mixture of step 3 and blended.

6. Sifted magnesium stearate through an ASTM #60 mesh sieve.

7. Lubricated the blend of step 5 by mixing with magnesium stearate of step 6.

8. Compressed the lubricated blend of step 7 into tablets.

An in vitro dissolution study was conducted, using the USP procedure and the following conditions:

Apparatus: USP type II.

Stirring speed: 100 rpm.

Dissolution medium: pH 4.0 citrate buffer.

Volume of dissolution medium: 500 ml.

Results are given in the following table:

| Time | Cumulative % of Drug Dissolved (Example No.) | | | | | | | |
|---|---|---|---|---|---|---|---|---|
| (hours) | (1B) | (3B) | (6) | (7) | (8) | (9) | (10) | (11) |
| 1 | 16.3 | 11 | 25 | 8.7 | 15 | 15.7 | 10 | 12.3 |
| 2 | 25 | 17 | 40 | 15 | 23 | 23 | 14 | 18.7 |
| 4 | 37.3 | 27 | 57 | 25 | 35 | 34.7 | 22 | 28.7 |
| 6 | 55.3 | 43 | 71 | 42.7 | 56 | 52.7 | 34 | 46.0 |
| 8 | 68.3 | 60 | 74 | 58.3 | 75 | 67 | 48 | — |
| 12 | 79.7 | 73 | 76 | 70.3 | 87 | 76.7 | 61 | 73.7 |
| 20 | 84 | 81 | 78 | 81 | 95 | 84 | 68 | 83.3 |
| 24 | 86 | 88 | 79 | 89.3 | 100 | 90 | 76 | 91.3 |

### Examples 13-17

### Ropinirole 2 mg Controlled Release Trilayer Tablets

| | mg/Tablet (Example No.) | | | | |
|---|---|---|---|---|---|
| Ingredient | (13) | (14) | (15) | (16) | (17) |
| Layer 1 (barrier layer) | | | | | |
| Hypromellose K 100 M | 40 | 40 | 38 | 38 | — |
| Glyceryl behenate | 15 | 27 | 30 | 30 | — |
| Cellulose acetate | — | — | — | — | 75 |
| Lactose monohydrate | 35 | 23 | 23 | 23 | — |
| Microcrystalline cellulose | — | — | — | — | 15 |
| Povidone K-30 | 7 | 7 | 6 | 6 | 7 |
| Colloidal silicon dioxide | 1.5 | 1.5 | 1.5 | 1.5 | 1.5 |
| Magnesium stearate | 1.5 | 1.5 | 1.5 | 1.5 | 1.5 |
| Layer total | 100 | 100 | 100 | 100 | 100 |
| Layer 2 (active layer) | | | | | |
| Ropinirole HCl | 2.28 | 2.28 | 2.28 | 2.28 | 2.28 |
| Hypromellose K 100M | 75 | — | 135 | 25 | 100 |
| Hypromellose K 15M | 20 | — | — | — | — |
| Hydrogenated castor oil | — | 75 | 10 | 100 | 25 |
| Stearic acid | — | 18 | — | — | — |
| Microcrystalline cellulose | 22.72 | 22.72 | 0.22 | 20.22 | 20.22 |
| Lactose DCL -21 | — | 27 | — | — | — |
| Hydroxypropyl cellulose M | 15 | — | — | — | — |
| Povidone K 30 | 10 | — | — | — | — |
| Water* | 1333 | — | — | — | — |
| Colloidal silicon dioxide | 3 | 3 | 1 | 1 | 1 |
| Magnesium stearate | 2 | 2 | 1.5 | 1.5 | 1.5 |
| Layer total | 150 | 150 | 150 | 150 | 150 |
| Layer 3 (barrier layer) | | | | | |
| Hypromellose K 100M | 45 | 45 | 40 | 40 | — |
| Glyceryl behenate | 25 | 25 | 27 | 27 | — |
| Cellulose acetate | — | — | — | — | 80 |
| Lactose monohydrate | 23 | 23 | 23 | 23 | — |
| Microcrystalline cellulose | — | — | — | — | 10 |
| Povidone K 30 | 5 | 5 | 7 | 7 | 7 |
| Colloidal silicon dioxide | 1 | 1 | 1.5 | 1.5 | 1.5 |
| Magnesium stearate | 1 | 1 | 1.5 | 1.5 | 1.5 |
| Layer total | 100 | 100 | 100 | 100 | 100 |
| Cumulative total | 350 | 350 | 350 | 350 | 350 |

*Evaporates during processing.

Example 13 manufacturing process:

Active layer (Layer 2):

1. Mix hypromellose K100M, hypromellose K15M, hydroxypropyl cellulose M and microcrystalline cellulose; and sift through an ASTM #40 mesh sieve.

2. Mix the contents from step 1 geometrically with ropinirole and sift through an ASTM #40 mesh sieve.

3. Prepare a 5% w/w aqueous solution of povidone and granulate the material of step 2 using this solution as a granulating agent.

4. Dry the granules of step 3 and sift through an ASTM #20 mesh sieve.

5. Sift colloidal silicon dioxide through an ASTM #40 mesh sieve and blend with the granules of step 4.

6. Sift magnesium stearate through an ASTM #60 mesh sieve and blend with the contents from step 5.

Barrier layers (Layers 1 and 3):

1. Mix hypromellose, glyceryl behenate, lactose monohydrate and povidone; and sift through an ASTM #40 mesh sieve.

2. Heat the mixture of step 1 to 65° C. to obtain granules, followed by cooling, and pass through an ASTM #40 mesh sieve.

3. Sift colloidal silicon dioxide through an ASTM #40 mesh sieve and blend with granules of step 2.

4. Sift magnesium stearate through an ASTM #60 mesh sieve and blend with the material from step 3.

Compression:

Load the lubricated blends active and barrier layers into the feed boxes of a triple rotary compressing machine and compress into trilayer tablets. First, add the composition of first barrier layer (Layer1) to the die cavity followed by pre-compression, then add active layer (Layer2) composition to the die cavity followed by pre-compression, then add second barrier layer (Layer3) composition and compress the layers into a trilayer tablet.

Example 14 manufacturing process:

Active layer (Layer 2):

1. Melt hydrogenated castor oil and stearic acid.

2. Disperse ropinirole uniformly in the molten material of step 1 with constant stirring and allow the molten mass to cool gradually.

3. Mix microcrystalline cellulose and lactose and sift through an ASTM #40 mesh sieve

4. Adsorb the semi-solid mass of step 2 onto the mixture of step 3 and sift through an ASTM #40 mesh sieve, followed by mixing.

5. Sift colloidal silicon dioxide through an ASTM #40 mesh sieve and blend with the material of step 4.

6. Sift magnesium stearate through an ASTM #60 mesh sieve and blend with the material from step 5.

Barrier layers (Layers 1 and 3): similar to the process described for Example 13.

Compression: similar to the process described for Example 13.

Example 15 manufacturing process:

Active layer (Layer 2):

1 Melt hydrogenated castor oil and disperse ropinirole uniformly in the molten material with constant stirring, then allow the molten mass to cool gradually.

2. Mix hypromellose and microcrystalline cellulose and sift through an ASTM #40 mesh sieve.

3. Adsorb the semi-solid mass of step 1 onto the mixture of step 2 and sift through an ASTM #40 mesh sieve, followed by mixing.

4. Sift colloidal silicon dioxide through an ASTM #40 mesh sieve and blend with the material of step 3.

5. Sift magnesium stearate through an ASTM #60 mesh sieve and blend with the material from step 4.

Barrier layers (Layers 1 and 3): similar to the process described for Example 13.

Compression: similar to the process described for Example 13.

Example 16 manufacturing process:

Active layer (Layer 2): similar to the process described for Example 15.

Barrier layers (Layers 1 and 3): similar to the process described for Example 13.

Compression: similar to the process described for Example 13.

Example 17 manufacturing process:

Active layer (Layer 2): similar to the process described for Example 15.

Barrier layers (Layers 1 and 3):

1. Mix cellulose acetate, microcrystalline cellulose and povidone, and sift through an ASTM #40 mesh sieve.

2. Sift colloidal silicon dioxide through an ASTM #40 mesh sieve and blend with the material of step 1.

3. Sift magnesium stearate through an ASTM #60 mesh sieve and blend with the material from step 2.

Compression: similar to the process described for Example 13.

### Examples 18-19

### Ropinirole 2 mg Controlled Release Bilayer Tablets

| | mg/Tablet | |
|---|---|---|
| Ingredient | Example 18 | Example 19 |
| Layer 1 | | |
| Ropinirole HCl | 0.285 | 1.14 |
| Microcrystaline cellulose | 142.215 | 77.36 |
| Povidone K 30 | — | 7 |
| Hypromellose K 15M | — | 45 |
| Glyceryl behenate | — | 15 |
| Sodium starch glycolate | 3 | — |
| Colloidal silicon dioxide | 2 | 2 |
| Magnesium stearate | 2.5 | 2.5 |
| Layer total | 150 | 150 |
| Layer 2 | | |
| Ropinirole HCl | 1.995 | 1.14 |
| Hypromellose K 15M | 135 | — |
| Hypromellose K 100M | — | 135 |
| Microcrystalline cellulose | 84.005 | 109.86 |
| Povidone K 30 | 25 | — |
| Water* | 500 | — |
| Colloidal silicon dioxide | 2 | 2 |
| Magnesium stearate | 2 | 2 |
| Layer total | 250 | 250 |
| Cumulative Total | 400 | 400 |

*Evaporates during processing.

Example 18 manufacturing process:

Layer 1:

1. Sift microcrystalline cellulose, sodium starch glycolate and colloidal silicon dioxide through an ASTM #40 mesh sieve, mix geometrically with ropinirole, and sift through an ASTM #40 mesh sieve.

2. Sift magnesium stearate through an ASTM #60 mesh sieve and blend with the material from step 1.

Layer 2:

1. Sift microcrystalline cellulose, hypromellose and povidone through an ASTM #40 mesh sieve, mix geometrically with ropinirole, and sift through an ASTM #40 mesh sieve.

2. Granulate the material from step 1, using water as a granulating agent.

3. Dry the granules obtained in step 2 and sift through an ASTM #20 mesh sieve.

4. Sift colloidal silicon dioxide through an ASTM #40 mesh sieve and blend with the material of step 3.

5. Sift magnesium stearate through an ASTM #60 mesh sieve and blend with the material from step 4.

Compression: Load the lubricated blends of both layers into the feed boxes of a rotary compression machine and compress into bilayer tablets. First, add the composition of Layer 1 to the die cavity followed by pre-compression, then add the Layer 2 composition and coMpress the layers into a bilayer tablet.

Example 19 manufacturing process:

Layer 1:

1. Sift microcrystalline cellulose, povidone, hypromellose and colloidal silicon dioxide through an ASTM #40 mesh sieve; and mix well.

2. Melt glyceryl behenate and disperse ropinirole uniformly in the molten material with constant stirring, then allow the molten mass to cool gradually.

3. Adsorb the semi-solid mass of step 2 on to the contents of step 1 and sift through an ASTM #40 mesh sieve, followed by mixing.

4. Sift magnesium stearate through an ASTM #60 mesh sieve and blend with the material from step 3.

Layer 2:

1. Mix hypromellose, microcrystalline cellulose and colloidal silicon dioxide and sift through an ASTM #40 mesh sieve.

2. Mix the material of step 1 with ropinirole and sift through an ASTM #40 mesh sieve.

3. Sift magnesium stearate through an ASTM #60 mesh sieve and blend with the material from step 2.

Compression: similar to the process described for Example 18.

### Examples 20-22

### Ropinirole 2 mg Controlled Release Multiparticulate Compositions

| | mg/Unit | | |
|---|---|---|---|
| Ingredient | Example 20 | Example 21 | Example 22 |
| Immediate Release (IR) Pellets | | | |
| Ropinirole HCl | 0.285 | 0.285 | — |
| Microcrystalline cellulose | 90.715 | 75 | — |
| Povidone K 30 | 9 | 9 | — |
| Mannitol | 50 | 15.715 | — |
| Methanol* | 100 | 100 | — |
| IR pellets total | 150 | 100 | — |
| Controlled Release (CR) Pellets 1 | | | |
| Ropinirole HCl | 1.995 | 0.9975 | 1.14 |
| Microcrystalline cellulose | 95.255 | 57.2525 | 112.36 |
| Povidone K 30 | 7 | 6 | 6 |
| Methanol* | 100 | 100 | 100 |
| Eudragit RS | 45 | — | — |
| Ethylcellulose | — | 35 | 30 |
| Triethyl citrate | 0.75 | 0.3 | 0.15 |
| Dibutyl sebacate | — | 0.45 | 0.35 |
| Water* | 405 | 81.667 | 100 |
| CR pellets 1 total | 150 | 100 | 150 |
| CR Pellets 2 | | | |
| Ropinirole HCl | — | 0.9975 | 1.14 |
| Microcrystalline cellulose | — | 71.2525 | 121.11 |
| Povidone K 30 | — | 7 | 7 |
| Methanol* | — | 100 | 100 |
| Eudragit RS | — | 20 | 20 |
| Triethyl citrate | — | 0.75 | 0.75 |
| Water* | — | 180 | 180 |
| CR pellets 2 total | — | 100 | 150 |
| Cumulative Total | 300 | 300 | 300 |

*Evaporates during processing.

Example 20 manufacturing process:

Immediate release pellets:

1. Mix microcrystalline cellulose and mannitol; and sift through an ASTM #40 mesh sieve.

2. Dissolve ropinirole in methanol; and add povidone with stirring until complete dissolution is obtained.

3. Granulate the material from step 1 using the solution of step 2 to obtain a damp mass, then extrude and spheronize using a spheronizer.

4. Dry the pellets of step 3 at 45° C. for 6-8 hours.

Controlled release pellets 1:

1. Sift microcrystalline cellulose through an ASTM #40 mesh sieve.

2. Dissolve ropinirole in methanol; and add povidone with stirring until complete dissolution is obtained.

3. Granulate the material from step 1 using the solution of step 2 to obtain a damp mass, then extrude and spheronize using a spheronizer.

4. Dry the pellets of step 3 at 45° C. for 6-8 hours.

5. Add triethyl citrate slowly to water with stirring until complete solution is obtained, and add Eudragit RS with stirring for 30 minutes to obtain a uniform dispersion.

6. Coat the pellets of step 4 with the dispersion of step 5 followed by drying to obtain controlled release pellets.

Formulation:

Mix the IR pellets and CR pellets 1 in a desired ratio, and fill into a hard gelatin capsule or compress into tablets together with suitable pharmaceutical excipients.

Example 21 manufacturing process:

Immediate release pellets: similar to the process described for Example 20.

Controlled release pellets 1:

1. Sift microcrystalline cellulose through an ASTM #40 mesh sieve.

2. Dissolve ropinirole in methanol and add povidone with stirring until complete dissolution is obtained.

3. Granulate the material from step 1 using the solution of step 2 to obtain a damp mass, then extrude and spheronize using a spheronizer.

4. Dry the pellets of step 3 at 45° C. for 6-8 hours.

5. Add ethylcellulose slowly to water with stirring to obtain a uniform dispersion; add slowly dibutyl sebacate with stirring for 30 minutes, and add slowly triethyl citrate into the vortex followed by stirring for 30 minutes.

6. Coat the pellets of step 4 with the aqueous dispersion of step 5 followed by drying to obtain controlled release pellets.

Controlled release pellets 2: similar to the process described for Example 20 for controlled release pellets 1.

Formulation:

Mix the IR pellets, CR pellets 1 and CR pellets 2 in a desired ratio, and fill into hard gelatin capsules or compress into tablets together with suitable pharmaceutical excipients.

Example 22 manufacturing process:

Controlled release pellets 1: similar to the process described for Example 21.

Controlled release pellets 2: similar to the process described for Example 20 controlled release pellets 1.

Formulation: similar to the process described for Example 21.

### Example 23

### Ropinirole 2 mg Controlled Release Multiparticulate Composition

| Ingredient | mg/Unit |
|---|---|
| CR Pellets 1 | |
| Non-pariel seeds | 120 |
| Ropinirole HCl | 1.14 |
| Povidone K 30 | 5 |
| HPMC 3-5 cp | 4 |
| Methanol* | 100 |
| Eudragit RS | 20 |
| Triethyl citrate | 0.75 |
| Water* | 180 |
| CR pellets 1 total | 150.89 |
| CR Pellets 2 | |
| Non-pariel seeds | 110 |
| Ropinirole HCl | 1.14 |
| Povidone K 30 | 6 |
| HPMC 3-5 cp | 3 |
| Methanol* | 100 |
| Ethyl cellulose | 30 |
| Dibutyl sebacate | 0.35 |
| Triethyl citrate | 0.15 |
| Water* | 100 |
| CR pellets 2 total | 150.64 |
| Cumulative Total | 301.53 |

*Evaporates during processing.

Manufacturing process:

Controlled release pellets 1:

1. Dissolve ropinirole in methanol with stirring; add povidone and HPMC into the vortex with stirring until complete dissolution is obtained.

2. Coat the non-pariel seeds using the solution from step 1, followed by drying to obtain drug layered pellets.

3. Add triethyl citrate slowly to water with stirring until complete dissolution is obtained, then add Eudragit RS with stirring for 30 minutes to obtain a uniform dispersion.

4. Coat the pellets of step 2 using the dispersion of step 3, followed by drying to obtain controlled release pellets.

Controlled release pellets 2:

1. Dissolve ropinirole in methanol with stirring and add povidone and HPMC into the vortex with stirring until complete dissolution is obtained.

2. Coat the non-pariel seeds using the solution from step 1 followed by drying to obtain drug layered pellets.

3. Add ethylcellulose slowly to water with stirring to obtain a uniform dispersion, add slowly dibutyl sebacate and stir for 30 minutes, then add slowly triethyl citrate into the vortex and stir for 30 minutes.

4. Coat the drug layered pellets of step 2 using the dispersion of step 3, followed by drying to obtain controlled release pellets.

Formulation:

Mix CR pellets 1 and CR pellets 2 in a desired ratio, then fill into hard gelatin capsules or compress into tablets together with suitable pharmaceutical excipients.

### Example 24

### Ropinirole 3 mg Controlled Release Multiparticulate Composition

| Ingredient | mg/Unit |
|---|---|
| IR Pellets | |
| Non-pariel seeds | 110 |
| Ropinirole HCl | 1.14 |
| Povidone K 30 | 6 |
| HPMC 3-5 cps | 4 |
| Methanol* | 100 |
| IR pellets total | 121.14 |
| CR Pellets 1 | |
| IR Pellets (above) | 121.14 |
| Eudragit RS | 20 |
| Triethyl citrate | 0.75 |
| Water* | 180 |
| CR pellets 1 total | 141.89 |
| CR Pellets 2 | |
| IR pellets (above) | 121.14 |
| Ethyl cellulose | 20 |
| Dibutyl sebacate | 0.35 |
| Triethyl citrate | 0.15 |
| Water* | 100 |
| CR pellets 2 total | 141.64 |
| Cumulative Total | 404.67 |

*Evaporates during processing.

Manufacturing process:

Immediate release pellets:

1. Dissolve ropinirole in methanol with stirring; add povidone and HPMC into the vortex with stirring until complete dissolution is obtained.

2. Coat the non-pariel seeds using the solution from step 1, followed by drying to obtain immediate release pellets.

Controlled release pellets 1:

1. Add triethyl citrate slowly to water with stirring until complete dissolution is obtained, and add Eudragit RS slowly with stirring for 30 minutes to obtain a uniform dispersion.

2. Coat the IR pellets using the dispersion of step 1, followed by drying to obtain controlled release pellets 1.

Controlled release pellets 2:

1. Add ethylcellulose slowly to water with stirring to obtain a uniform dispersion; add slowly dibutyl sebacate with stirring for 30 minutes, and add slowly triethyl citrate into the vortex with stirring for 30 minutes.

2. Coat the IR pellets using the dispersion of step 1, followed by drying to obtain controlled release pellets 2.

Formulation:

Mix IR pellets, CR pellets 1 and CR pellets 2 in a desired ratio, then fill into a hard gelatin capsule or compress into a tablet together with suitable pharmaceutical excipients.

### Example 25

### Ropinirole 2 mg Controlled Release Tablets

| Ingredient | mg/Tablet |
|---|---|
| Tablets | |
| Ropinirole hydrochloride* | 2.28 |
| HPMC K 100 M | 240 |
| HPMC K 15 M | 60 |
| Povidone K 90 | 75 |
| Lactose DCL 21 | 18.72 |
| Colloidal silicon dioxide | 2 |
| Magnesium stearate | 2 |
| Total | 400 |
| CR Coating | |
| Ethyl cellulose 10 cp | 12.31 |
| Opadry OY-58900 (White) | 18.46 |
| Triethyl citrate | 1.23 |
| Dichloromethane** | 197.4 |
| Isopropyl alcohol** | 197.4 |
| Cumulative Total | 432 |
| Film Coating | |
| Opadry 02B14941 (Pink) | 12.96 |
| Water** | 95.04 |
| Cumulative Total | 444.96 |

*Equals 2 mg of ropinirole, particle size distribution of ropinirole hydrochloride: D90 < 50 μm.
**Evaporates during processing.

Manufacturing process:

1. Added ropinirole hydrochloride, part of the colloidal silicon dioxide and part of the lactose and sifted together through an ASTM #30 mesh sieve, then rinsed the sieve with a small amount of lactose.

2. Mixed povidone and a part of the lactose and sifted along with the material of step 1 through an ASTM #30 mesh sieve.

3. Blended the mixture of step 2 for 20 minutes in a double cone blender (DCB).

4. Mixed remaining lactose and HPMC K 15M, and sifted together with material of step 3 through an ASTM #30 mesh sieve, then blended in a DCB for 20 minutes.

5. Sifted the material of step 4 with HPMC K 100M through an ASTM #30 mesh sieve and blended for 20 minutes in a DCB.

6. Added remaining colloidal silicon dioxide and 3/4 of the magnesium stearate to the blend of step 5 and blended for 5 minutes.

7. Compacted the blend of step 6 and milled through a 5 mm screen at slow speed, knives forward, then sifted through an ASTM #22 mesh sieve.

8. Transferred the blend of step 7 into a DCB and added the remaining magnesium stearate, and blended for 5 minutes.

9. Compressed the blend of Step 8 into tablets using 12.5×7 mm capsule shaped punches.

CR Coating:

10. Dissolved ethyl cellulose and triethyl citrate in a mixture of isopropyl alcohol and dichloromethane and added Opadry OY-58900 (white) and stirred for 45 minutes.

11. Coated the tablets of step 9 with the dispersion of step 10 to produce an 8% weight gain.

Film Coating: 12. Coated the tablets of Step 11 with an aqueous dispersion of Opadry 2B 14941 pink to produce a 3% weight gain.

The tablets were analyzed to determine drug content uniformity, for which 10 samples were collected throughout the batch and analyzed to determine the content of active ingredient. The results were found to be acceptable and are given below, where the values are percentages of the label drug content.

| Sam- | Tablet | | | | | | | | | |
|---|---|---|---|---|---|---|---|---|---|---|
| ple | 1 | 2 | 3 | 4 | 5 | 6 | 7 | 8 | 9 | 10 |
| Assay | 96.7 | 95.2 | 96.2 | 93.6 | 96.4 | 95.9 | 93.6 | 95.5 | 96 | 95.9 |

The average assay was 95.5%, and the relative standard deviation was 1.1%.

The tablets were also analyzed to determine the dissolution profile, along with a commercial controlled release reference product: Requip® Modutab from GlaxoSmithKline, Batch No. X1367 (trilayer tablets), obtained from Latvia.

In vitro dissolution study conditions:

Apparatus: USP type II.

Stirring speed: 100 rpm.

Dissolution medium: pH 4.0 citrate buffer and pH 6.8 phosphate buffer.

Volume of dissolution medium: 500 ml.

Results are given in the following table.

| | Cumulative % of Drug Dissolved | | | |
|---|---|---|---|---|
| Time | pH 4.0 Citrate Buffer | | pH 6.8 Phosphate Buffer | |
| (hours) | Reference | Example 25 | Reference | Example 25 |
| 1 | 11 | 6 | 11 | 9 |
| 2 | 18 | 18 | 18 | 19 |
| 4 | 30 | 36 | 29 | 35 |
| 8 | 51 | 58 | 50 | 55 |
| 12 | 71 | 71 | 67 | 72 |
| 16 | 86 | 81 | 81 | 81 |
| 20 | 96 | 90 | 91 | 87 |
| 24 | 102 | 96 | 98 | 91 |

The calculated f₂ value for the Example 25 tablets is 66 in pH 4 buffer, and 69 in pH 6.8 buffer.

### Example 26

### Pharmacokinetic Study

The product of Example 25, and Requip®Modutab ropinirole 2 mg controlled release tablets from GlaxoSmithKline as a reference, were used in a randomized, open-label, balanced, two-treatment, two-period, two-sequence, single oral dose, crossover bioequivalence study in healthy adult male, human subjects under the fed condition.

A total of 18 healthy human adult male subjects were involved in the study and housed from at least 11 hours prior to drug administration until 24 hours after drug administration. In each period, after an overnight fast of at least 10 hours, the subjects were dosed either with the test or reference product accompanied by about 240 ml of drinking water according to the randomization schedule, after 30 minutes from the start of a high fat breakfast. Along with drug administration, domperidone tablets 2×10 mg were given in three doses (1^{5t} dose together with ropinirole drug administration, 2^{nd} dose at 8 hours after ropinirole administration, and the 3^{rd} dose at 16 hours after ropinirole administration) as a prophylactic measure to prevent nausea and vomiting caused by the ropinirole drug. Blood samples were collected before ropinirole dosing and at 1, 2, 3, 4, 5, 6, 7, 8, 9, 10, 11, 12, 14, 16, 24, and 36 hours post-dosing, and plasma concentrations of ropinirole were assayed by a validated LC-MS/MS method. There was a washout period of at least 7 days between each drug administration.

The phamiacokinetic parameters for ropinirole were calculated by standard non-compartmental methods and are summarized in the following table.

| Pharmacokinetic | | |
|---|---|---|
| Parameter | Example 25 | Reference |
| Cₘₐₓ (ng/ml) | 1884.5 | 1790.1 |
| AUC₀₋ₜ (ng•hour/ml) | 30308.9 | 31929 |
| AUC_{0-∞} (ng•hour/ml) | 31838.8 | 34753.2 |
| Tₘₐₓ (hours) | 9.5 | 10 |
| t½ (hours) | 5.81 | 6.83 |
| Kₑₗ (hr⁻¹) | 0.12 | 0.1 |

Kₑₗ is a first order rate constant for elimination of drug from the body, including the elimination by excretion and metabolism.

These results demonstrate that the tested compositions of the present invention are bioequivalent with the reference commercial product.

1. A controlled release monolithic tablet or multiparticulate pharmaceutical dosage form containing ropinirole, from which ropinirole completely dissolves over a period of more than 12 hours following immersion in an aqueous fluid.

2. The controlled release dosage form of claim 1, wherein ropinirole completely dissolves over a period of more than 16 hours.

3. The controlled release dosage form of claim 1, wherein ropinirole completely dissolves over a period of more than 20 hours.

4. The controlled release dosage form of claim 1, wherein ropinirole completely dissolves over a period of more than 24 hours.

5. The controlled release dosage form of claim 1, wherein not more than about 30 percent of ropinirole dissolves within about 1 hour following immersion.

6. The controlled release dosage form of claim 1, wherein about 40 percent to about 70 percent of ropinirole dissolves within about 8 hours to about 10 hours following immersion.

7. The controlled release dosage form of claim 1, wherein about 50 percent to about 80 percent of ropinirole dissolves within about 12 hours to about 14 hours following immersion.

8. The controlled release dosage form of claim 1, wherein about 70 percent to about 90 percent of ropinirole dissolves within about 16 hours to about 20 hours after immersion.

9. The controlled release dosage form of claim 1, comprising about 20 to about 95 percent by weight of one or more polymers.

10. The controlled release dosage form of claim 1, comprising about 35 to about 85 percent by weight of one or more polymers contained in a tablet or multiparticulate matrix.

11. The controlled release dosage form of claim 1, comprising about 50 to about 75 percent by weight of one or more polymers contained in a tablet or multiparticulate matrix.

12. The controlled release dosage form of claim 9, wherein a polymer comprises a derivative of cellulose.

13. The controlled release dosage form of claim 9, wherein a polymer comprises a vinylpyrrolidone polymer.

14. The controlled release dosage form of claim 9, wherein a portion of a polymer content is present in a coating.

15. The controlled release dosage form of claim 14, wherein a coating modifies dissolution of ropinirole into an aqueous fluid.

16. The controlled release dosage form of claim 14, wherein a coating is an enteric coating.

17. The controlled release dosage form of claim 1, which is a monolithic tablet having a polymer coating.

18. The controlled release dosage form of claim 1, which is a capsule containing a plurality particles, at least a portion of particles having a polymer coating.

19. The controlled release dosage form of claim 18, wherein a portion of particles has no coating, and another portion has a coating.

20. The controlled release dosage form of claim 18, wherein different portions of coated particles have different coatings.

21. The controlled release dosage form of claim 1, wherein a source of ropinirole has a particle size distribution with D₉₀ less than about 100 μm.

22. The controlled release dosage form of claim 1, wherein a source of ropinirole has a particle size distribution with D₉₀ less than about 50 μm.

23. The controlled release dosage form of claim 1, wherein a source of ropinirole has a particle size distribution with D₉₀ less than about 20 μm.
